(19) United States
(12) Patent Application Publication
(10) Pub. No.: US 2018/0330812 A1
(43) Pub. Date: Nov. 15, 2018

(54) SYSTEM AND METHOD FOR MANAGING AND MONITORING A COMPLIANCE PROGRAM FOR A PLURALITY OF HEALTHCARE ENTITIES

(71) Applicant: First Healthcare Compliance, LLC, Wilmington, DE (US)

(72) Inventors: Julie Sheppard, Wilmington, DE (US); Loris Miller, Kennett Square, PA (US); Jill Kristin Brooks, Wilmington, DE (US)

(21) Appl. No.: 15/774,201

(22) PCT Filed: Nov. 9, 2016
(86) PCT No.: PCT/US16/61136

§ 371 Date: May 7, 2018

Related U.S. Application Data
(60) Provisional application No. 62/252,769, filed on Nov. 9, 2015.

(51) Int. Cl. G16H 40/20; G06Q 30/00

(52) U.S. Cl. CPC G16H 40/20; G06F 3/0482; G06Q 30/018

(57) ABSTRACT

Embodiments are directed to a system and method for managing and monitoring a compliance program for a healthcare entity, and managing and monitoring compliance programs for a plurality of healthcare entities. Embodiments disclosed herein provide a healthcare compliance portal that compiles and organizes the necessary laws and regulations for healthcare entities, allowing healthcare entities to identify and comply with the required laws and regulations. Billing entities as well as any other type of entity that may oversee or otherwise be affiliated with one or more healthcare providers may also utilize the healthcare compliance portal to determine compliance and/or identify compliance issues among one or more providers for which the billing entity processes bills, payments, vouchers, and the like.

### CROSS-REFERENCE TO RELATED APPLICATIONS

This application claims priority to U.S. Provisional Patent Application No. 62/252,769 filed on Nov. 9, 2015, the contents of which are herein incorporated by reference in their entirety

### BACKGROUND

Healthcare entities, such as medical practices, today are faced with the daunting task of complying with a multitude of laws and regulations administered by several federal agencies. Understanding and complying with these laws and regulations can be an expensive and time-consuming process, requiring the services of multiple consultants, each with a specific area of expertise.

Thus, there is a need for a streamlined system and method to compile and organize the necessary laws and regulations for healthcare entities, allowing healthcare entities to identify and comply with the required laws and regulations. Moreover, there is a need for the system and method to be manageable, efficient, and affordable for healthcare entities.

There is also a need for the system to be utilizable by an entity that oversees or is affiliated with one or more healthcare entities, allowing the entity to identify compliance issues with the one or more healthcare entities.

### SUMMARY

Embodiments disclosed herein are directed to a system and method for managing and monitoring a compliance program for a healthcare entity. Additional embodiments are directed to managing and monitoring a compliance program for a plurality of healthcare entities.

In an embodiment, a system and a computer-implemented method for managing and monitoring a compliance program for a healthcare entity, the healthcare entity comprised of (i) one or more supervisors, (ii) one or more employees, and (iii) one or more vendors, are provided. The one or more employees and the one or more vendors are required to comply with one or more compliance standards.

The system, according to an embodiment, comprises a portal comprising one or more servers operating over a network configured to interconnect one or more of the one or more supervisors, the one or more employees, and the one or more vendors. The one or more servers are configured to implement the method steps of: receiving input relating to one or more of employee data and vendor data; receiving input relating to the one or more compliance standards; compiling a compliance profile for each of the one or more employees and the one or more vendors for whom input is received; and conveying, through a graphical user interface (GUI), the compliance profile to the one or more supervisors and a corresponding one of the one or more employees and the one or more vendors, the compliance profile indicating compliance measures for the one or more compliance standards applicable to the one of the one or more employees and the one or more vendors.

In another embodiment, a system is provided for managing and monitoring a compliance program for a healthcare entity. The system comprises a portal comprising one or more servers operating over a network configured to interconnect one or more of one or more supervisors, one or more employees, and one or more vendors. The one or more servers are configured to: receive input relating to one or more of healthcare entity data, employee data, and vendor data; receive input relating to the one or more compliance standards from a system administrator; generate one or more queries for each of the one or more supervisors, the one or more employees, and the one or more vendors, the queries relating to the one or more compliance standards for corresponding ones of the one or more supervisors, the one or more employees, and the one or more vendors; receive inputs in response to the one or more queries from the one or more supervisors, the one or more employees, and the one or more vendors; compile a healthcare entity compliance profile comprising a summary of compliance measures for the one or more compliance standards applicable to the one or more supervisors, the one or more employees, and the one or more vendors; and convey, through a graphical user interface (GUI), the healthcare entity compliance profile to the one or more supervisors.

In yet another embodiment, a system is provided for monitoring a compliance program by a billing entity for a healthcare entity. The system comprises a portal comprising one or more servers operating over a network configured to interconnect one or more of one or more supervisors, one or more employees, and one or more vendors. The one or more servers are configured to: compile a compliance profile for each of the one or more employees and the one or more vendors, the compliance profile indicating compliance measures for the one or more compliance standards applicable to the one of the one or more employees and the one or more vendors; compile a healthcare entity compliance profile for the healthcare entity, the healthcare entity compliance profile indicating a summary of the compliance measures for the one or more compliance standards applicable to the one or more employees and the one or more vendors and compliance issues for the healthcare entity; and convey, through a graphical user interface (GUI), to the billing entity one or more of (i) the compliance profile for each of the one or more employees and the one or more vendors and (ii) the healthcare entity compliance profile.

Additional features and advantages are apparent from the following detailed description that proceeds with reference to the accompanying drawings.

### BRIEF DESCRIPTION OF THE DRAWINGS

The foregoing and other aspects of the invention are best understood from the following detailed description when read in connection with the accompanying drawings. The drawings depict embodiments solely for the purpose of illustration; it should be understood, however, that the disclosure is not limited to the specific instrumentalities disclosed. Included in the drawings are the following Figures:

FIG. 1 is a block diagram illustrating system components for managing and monitoring a compliance program for a healthcare entity, according to an embodiment;

FIG. 2 is a flow chart illustrating a data flow among various entities for managing and monitoring a compliance program for a healthcare entity, according to an embodiment;

FIGS. 3A-3J are a series of screenshots of a graphical user interface for a system for managing and monitoring a compliance program for a healthcare entity, according to embodiments; and

FIG. 4 is a screenshot of an exemplary employee graphical user interface, according to an embodiment.

### DETAILED DESCRIPTION

Embodiments are directed to a system and method for managing and monitoring a compliance program for a healthcare entity, and managing and monitoring compliance programs for a plurality of healthcare entities. Embodiments disclosed herein provide a healthcare compliance portal that compiles and organizes the necessary laws and regulations for healthcare entities, allowing healthcare entities to identify and comply with the required laws and regulations. The portal also allows for an entity to be provided with information necessary to determine compliance and/or compliance issues for one or more healthcare entities.

According to an embodiment, the healthcare compliance portal disclosed herein is comprised of compliance zones, in which various regulations are organized. Each compliance zone may be supported by training, support material, and technology that collects data pertaining to a compliance status of the particular healthcare entity and the relevant individuals and/or vendors. Moreover, the compliance zones are tailored and specific to the particular healthcare entity; i.e., dependent upon the type of healthcare entity and the associated compliance laws and regulations to which it must adhere.

According to an embodiment, some data for the healthcare compliance portal disclosed herein may be entered manually by an administrator of the healthcare entity or by an administrator of the portal, while other data may be automatically collected. Profiles and/or reports may be generated to assist the healthcare entity in identifying areas requiring remediation. The profiles and/or reports may also to be used for records as evidence of a compliance program, which may be required by one or more federal agencies.

The healthcare compliance portal disclosed herein may be adapted and used for a regulated industry in the healthcare field that is required to comply with various regulations and/or laws, either internal or as required by one or more agencies. Such entities include, but are not limited to, physical therapy practices, dental practices, nursing facilities, rehabilitation facilities, and any provider or specialty in the healthcare field. Moreover, billing entities as well as any other type of entity that may oversee or otherwise be affiliated with one or more healthcare providers may also utilize the healthcare compliance portal disclosed herein. A billing entity may utilize the portal to determine compliance among one or more providers for which the billing entity processes bills, payments, vouchers, and the like.

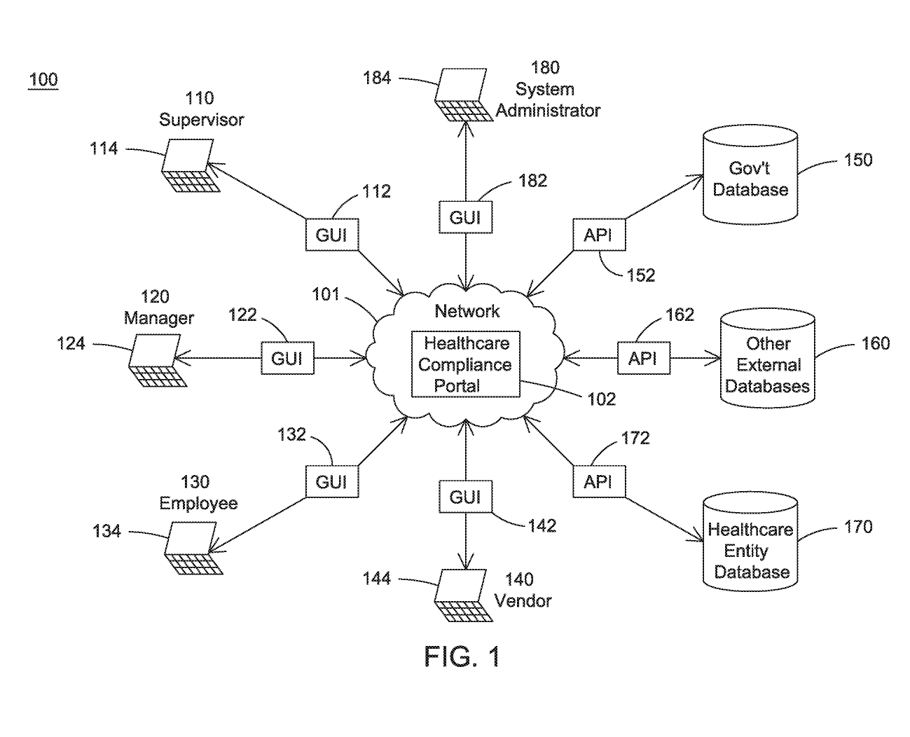

FIG. 1 is a block diagram 100 illustrating system components for managing and monitoring a compliance program for a healthcare entity, according to an embodiment. A healthcare compliance portal 102, for compiling and organizing the necessary laws and regulations for a healthcare entity, may operate in a cloud computing environment (e.g., network 101) and may be comprised of one or more servers, processors, memory media, and computer code, and may also include one or more databases and other computer components and code necessary for storing and utilizing information for managing and monitoring the compliance program for the healthcare entity according to various embodiments provided herein.

FIG. 1 provides an overview of typical ways users can access the healthcare compliance portal 102. Supervisor 110, manager 120, employee 130, vendor 140, and system administrator 180 may access the healthcare compliance portal 102 through a graphical user interface (GUI) 112, 122, 132, 142, and 182, respectively, with a respective computing device 114, 124, 134, 144, and 184.

The computing devices 114, 124, 134, 144, and 184 may include, but are not limited to, a traditional desktop computer, a notebook computer, a tablet, a mobile device, a smartphone, a terminal, or the like. Additional supervisors, managers, employees, vendors, and/or system administrators may also be included and allowed access to the healthcare compliance portal 102.

In an embodiment, various access levels are assigned to users of the portal 102 within a particular healthcare entity. For example, a supervisor 110 may have access to and control of the entire contents of the portal 102, while a manager 120 has access to and control of certain portions of the data, and an employee 110 has access to limited portions pertaining to the particular employee 110. The access levels may vary between healthcare entities and may be established depending upon the type or particular entity.

Additionally, information may be communicated directly to and from the healthcare compliance portal 102 without human operator interaction through an electronic data interchange (EDI) or other application programming interface (API). For example, a government database 150, other external databases 160, and a healthcare entity database 170 may communicate with the healthcare compliance portal 102 and its embedded software through the use of corresponding EDIs/APIs 152, 162, and 172. In one embodiment, the healthcare entity database 170 comprises data specific to the healthcare entity utilizing the portal 102 and to which the supervisor 110, manager 120, employee 130, and vendor 140 belong or are affiliated. In an embodiment, the healthcare entity database 170 comprises data relating to employee data, vendor data, and compliance standards and compliance profiles as further described in detail below.

According to an embodiment, a supervisor 110 may monitor compliance at multiple physical locations. For example, if a healthcare entity has a corporate headquarters that oversees several clinical locations, the supervisor 110 may view the compliance status of each of those locations. In one embodiment, the headquarters is one “account” and each of their locations are independent “accounts” with codes, for example, that identify the particular locations. In another embodiment, the entire hierarchy of the headquarters and associated locations may be considered as one “account,” recognizing the hierarchy with any number of subaccounts that can be different types of businesses (for example, one headquarters that owns two physical therapy locations, a billing company, and a hospital).

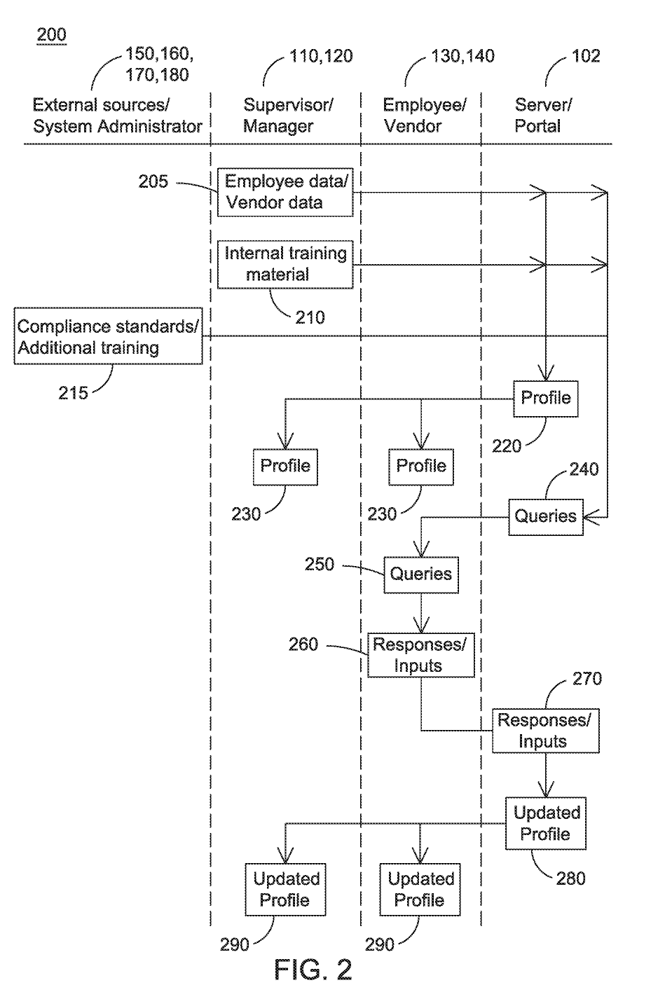

FIG. 2 is a flow chart 200 illustrating a data flow among various parties for managing and monitoring a compliance program for a healthcare entity, according to an embodiment. In an embodiment, the healthcare entity is comprised of one or more supervisors/managers 110, 120; one or more employees 130; and one or more vendors 140. The one or more employees 130 and the one or more vendors 140 are required to comply with one or more compliance standards. In an embodiment, the one or more supervisors may comprise the supervisor 110 and/or the manager 120, or a plurality thereof. In an embodiment, the healthcare entity is comprised of a hierarchy of supervisors 110, managers 120, and employees 130, and the healthcare entity utilizes one or more vendors 140 for performing services. In an alternate embodiment, the healthcare entity may include one supervisor 110 and a plurality of employees 130. Other combination of the parties in a healthcare entity may be envisioned and utilized in accordance with embodiments provided herein.

With reference to FIG. 2, the supervisor 110 and/or the manager 120 may provide employee data and/or vendor data to the healthcare compliance portal 102 (205). This data may include, but is not limited to, identification information, position information (i.e., a role or duties for one or more employees 130), vendor type, etc. The various data may be imported from a spreadsheet or the like by the supervisor 110 and/or the manager 120.

The supervisor 110 and/or the manager 120 may also provide internal training materials to the healthcare compliance portal 102 (210). The internal training materials may be specific to the particular healthcare entity, the employees 130, and/or the vendors 140.

The necessary laws and regulations for the healthcare entity (i.e., the compliance standards) may be provided by external sources, such as the various databases 150, 160, 170, or as inputted by the system administrator 180 (215). Additional training materials may also be provided. In an embodiment, the system administrator 180 is not affiliated with a particular healthcare entity, but instead oversees and maintains the healthcare compliance portal 102 and, as such, inputs updated laws and regulations that may pertain to a particular healthcare entity. Moreover, the system administrator 180 may oversee and maintain a healthcare compliance portal 102 for a plurality of various different healthcare entities.

With the inputs relating to employee data, vendor data, internal training, the necessary laws and regulations (i.e., the compliance standards), and/or the additional training materials, the healthcare compliance portal 102, according to one embodiment, compiles a compliance profile for each of the one or more employees 130 and the one or more vendors 140 (220). The compliance profile indicates compliance measures for the one or more compliance standards applicable to the employees 130 and the vendors 140. For example, the compliance profile for a specific employee 130 may indicate those compliance standards the employee has met and those that are not yet satisfied.

The healthcare compliance portal 102 conveys the compliance profile to the supervisor 110, the manager 120, and/or the corresponding employee 130 or vendor 140 (230). The conveyance of the compliance profile may be through a GUI specific to the user (e.g., a GUI for the supervisor, a GUI for the manager, a GUI for the employee, and a GUI for the vendor, with each GUI displaying information pertinent to the particular user). In an additional embodiment, an email message or a URL link (via an email message, a text message, or a pop-up window, for example) is provided for accessing the compliance profile. In an embodiment, only the supervisor 110 and/or manager 120 are provided with the compliance profiles for employees 130 and vendors 140.

In an embodiment, the healthcare compliance portal 102 generates one or more queries relating to the compliance standards for the employee 130 or the vendor 140 (240), and conveys (through, for example, the GUI, an email message, or a URL link) the one or more queries, to the employee 130 or the vendor 140 (250). The queries may include the internal training and/or additional training as provided by a government database, for example. The queries, in an embodiment, may comprise a series of questions for determining compliance for the particular employee 130 or vendor 140, as provided by, for example, the system administrator 180.

The employee 130 or the vendor 140 provides responses and/or inputs in response to the queries (260) through GUIs (e.g., the employee GUI 132 or the vendor GUI 142), which are received by the healthcare compliance portal 102 (270). With the responses/inputs, the healthcare compliance portal 102 compiles an updated compliance profile for the employee 130 or the vendor 140 (280).

The healthcare compliance portal 102 conveys the updated compliance profile to the supervisor 110, the manager 120, and/or the corresponding employee 130 or vendor 140 (290). In some embodiments, the updated compliance profile is provided to the supervisor 110 or the manager 120. The updated compliance profile indicates updated compliance measures for the compliance standards applicable to the employee or the vendor. For example, a particular employee 130 may have satisfied a compliance standard by answering a series of questions, and thus the employee's updated compliance profile would accordingly indicate this new status.

The updated compliance profile may be sent at predetermined intervals, such as weekly or monthly, to the supervisor 110 and/or the manager 120. Users may set preferences dictating desired intervals for receiving any updates. Alternatively, the predetermined intervals for receiving updates may be established by the system administrator 180 or a supervisor 110.

Additionally, according to another embodiment, employees 130 and/or vendors 140 may regularly (e.g., weekly, monthly, quarterly, annually) be required to complete training. Thus, an employee 130 and/or vendor 140 may be advised that additional compliance measures need to be satisfied. The compliance profile or a window in a GUI may provide access to the additional compliance measures for the particular employee 130 and/or vendor 140. Once the compliance measures are satisfied and sent to the healthcare compliance portal 102, the portal 102 routinely or automatically updates the appropriate compliance profile, indicating satisfaction of the compliance measures. Additionally, if the compliance measures are not satisfied, the updated compliance profile also reflects such a status (e.g., indicating the standards not yet satisfied).

According to an embodiment, the healthcare compliance portal 102 compiles a healthcare entity compliance profile for the healthcare entity. The healthcare entity compliance profile comprises a summary of compliance measures for the supervisor 110/manager 120, the employees 130, and the vendors 140 comprising the healthcare entity, allowing a user (e.g., the supervisor 110 and/or the manager 120) to identify overall compliance for the entity. This also allows a user to easily identify employees 130 and/or vendors 140 that are not in compliance, as well as the non-compliant areas. According to an embodiment, the supervisor 110 and/or manager 120 answers queries relating to compliance standards of the overall healthcare entity, which is also incorporated in the healthcare entity compliance profile.

Additionally, according to an embodiment, a user (e.g., the supervisor 110 and/or the manager 120) may automatically receive the various profiles at a predetermined interval, e.g., weekly, monthly. The user may also, through for example the supervisor GUI 112 or the manager GUI 122, view the profile when desired.

The healthcare entity compliance profile may also include historical data, indicating employees' and vendors' histories of responding to queries and satisfying one or more compliance standards. The historical data may also reflect a history of various healthcare compliance standards for the overall healthcare entity.

In an embodiment, a billing entity utilizes the healthcare entity compliance profile to determine if the healthcare entity is in compliance with all or a subset of applicable compliance standards. The billing entity may receive the healthcare entity compliance profile through an electronic message, a dedicated billing GUI, and/or a secure URL link. Moreover, the billing entity may receive the profile at a predetermined interval, e.g., monthly.

According to an additional embodiment, the healthcare compliance portal 102 may utilize government information as an additional layer of determining compliance for an employee 130 and/or a vendor 140. In an embodiment, the healthcare compliance portal 102 accesses or receives government data related to one or more employees 130 and/or one or more vendors 140. The data may be received from a government database 150, and the data may be compared to the employee data to determine compliance with one or more measures. In another embodiment, the data received from the government database 150 may indicate a status relating to a particular employee 130, where the status is a direct indicator of compliance for an employee 130 and/or vendor 140. For example, the government database 150 may provide a status or data on immigration, national security, criminal records, and the like.

In an embodiment, one or more vendors 140 are not provided with a vendor GUI and do not have access to the healthcare compliance portal 102. Rather, data relating to the one or more vendors 140 is inputted and/or imported for internal use by the supervisor 110 and/or the manager 120.

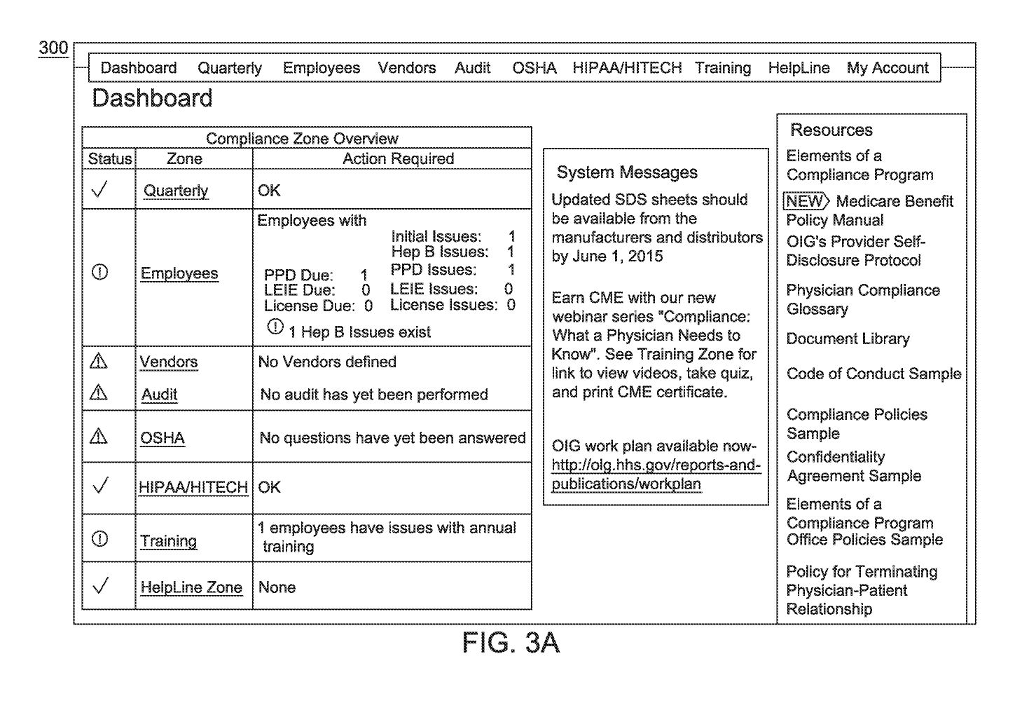

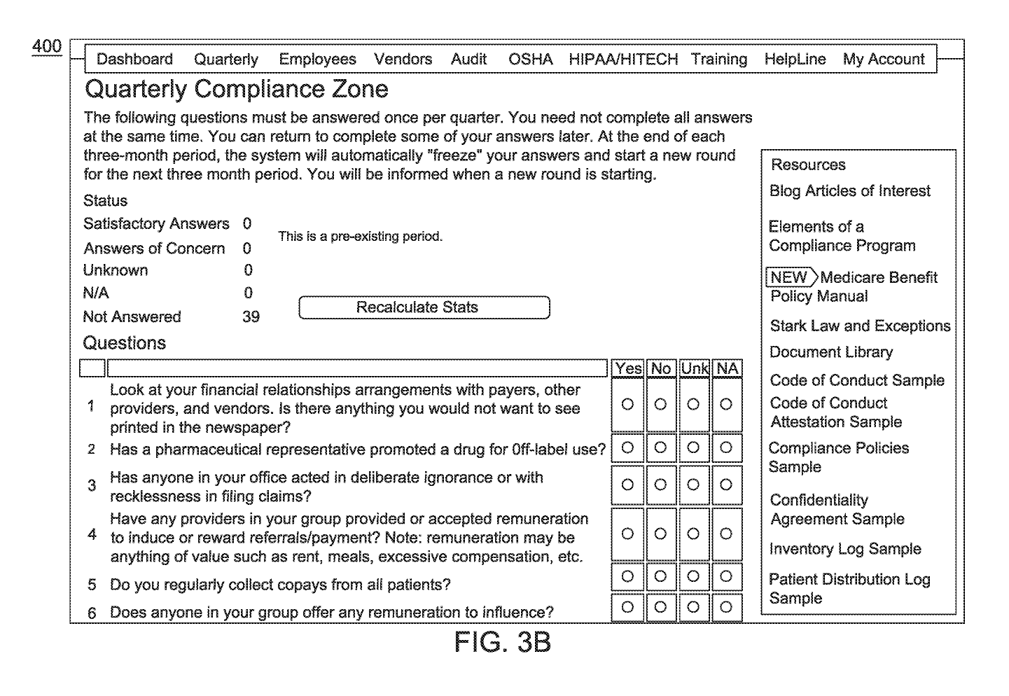

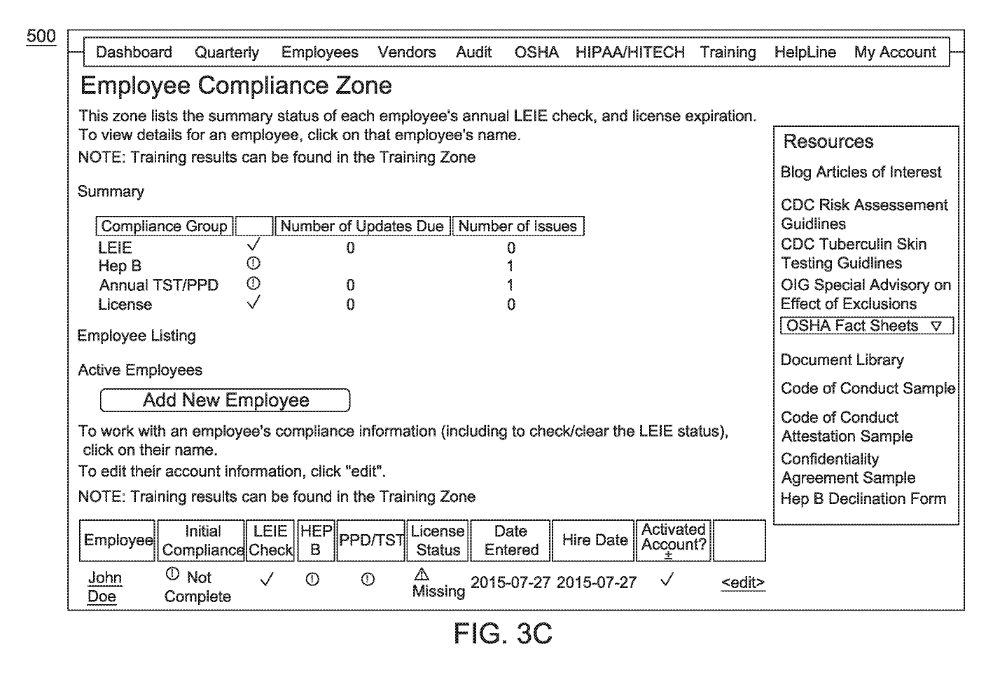

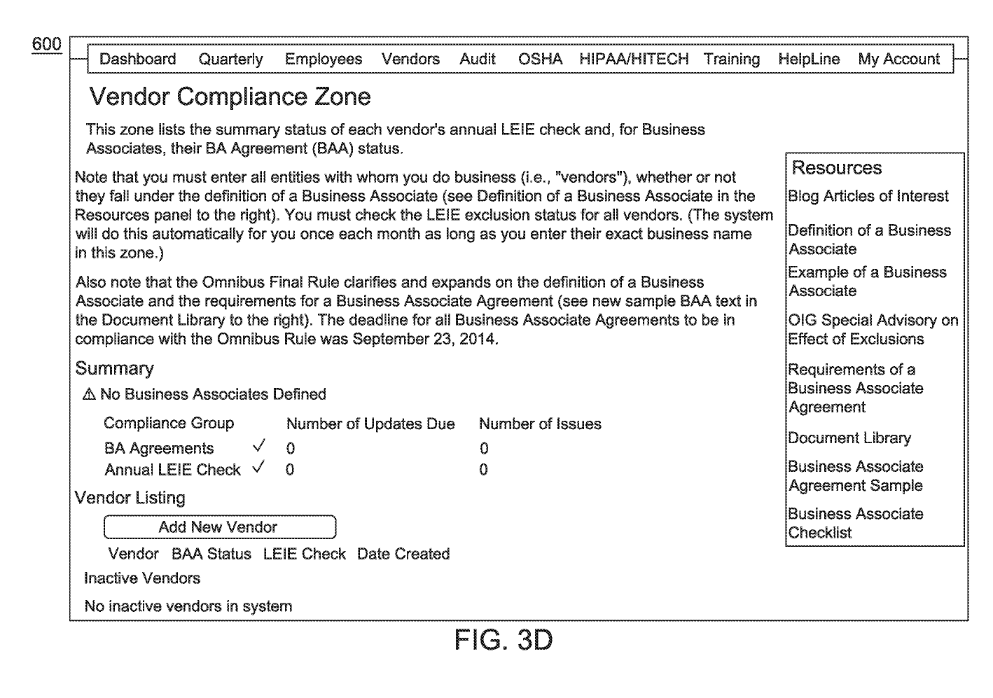

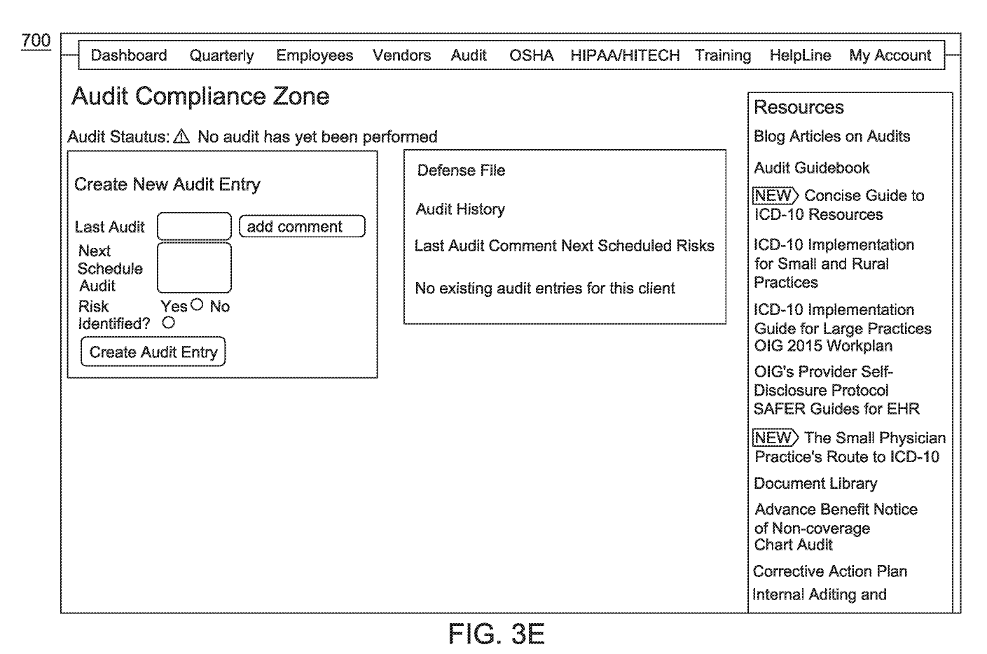

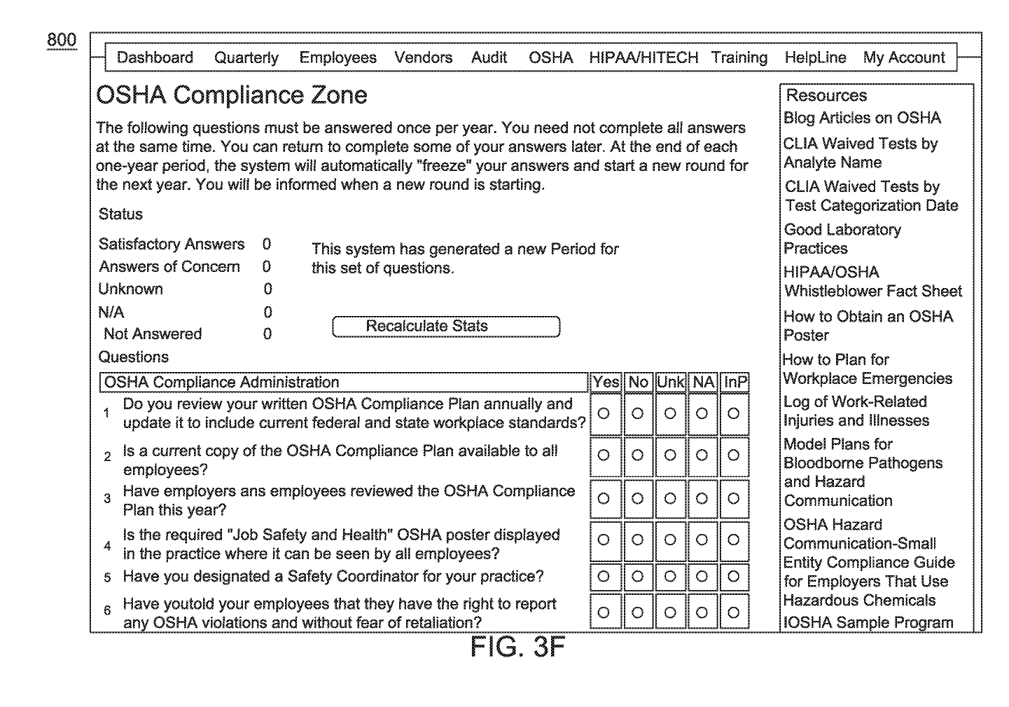

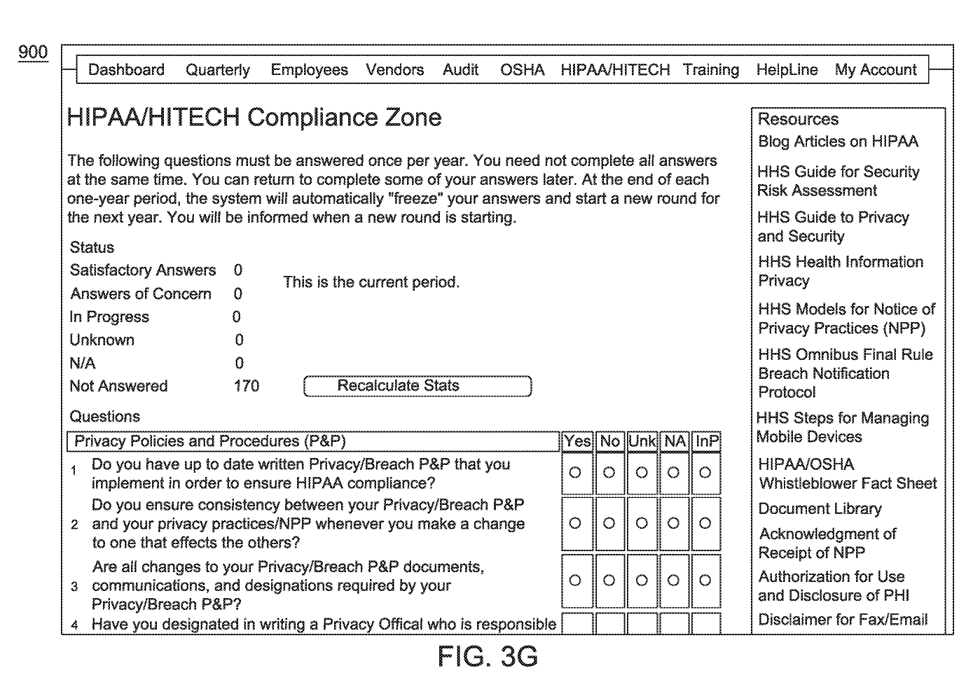

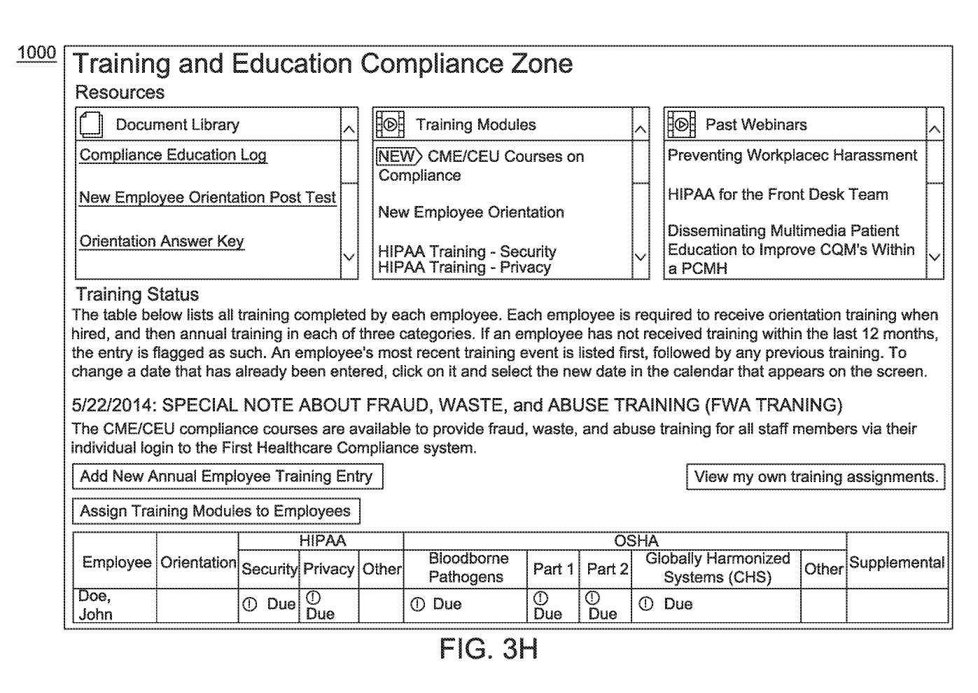

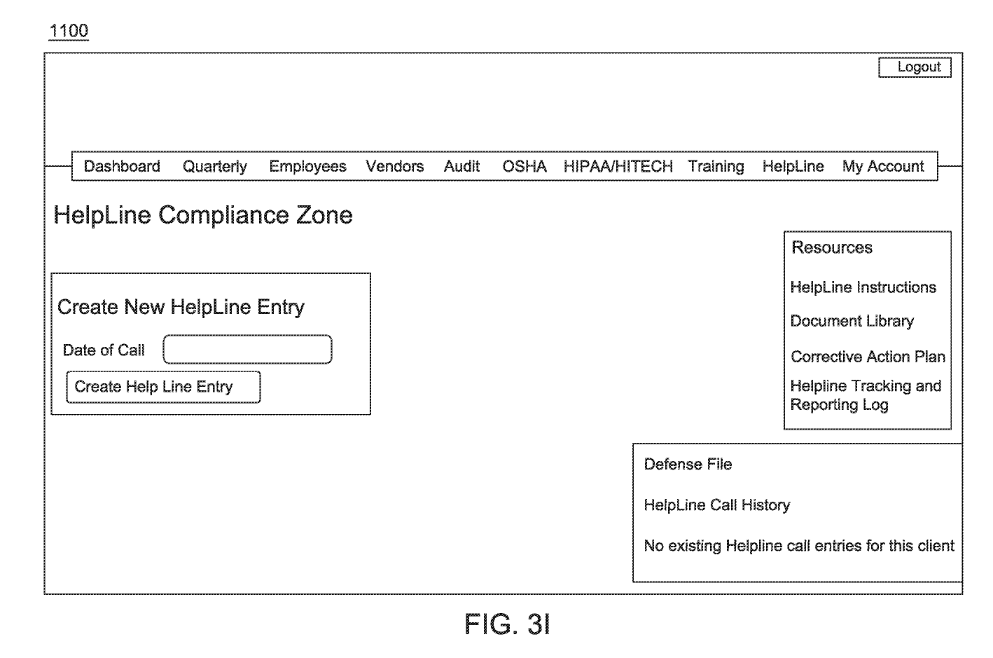

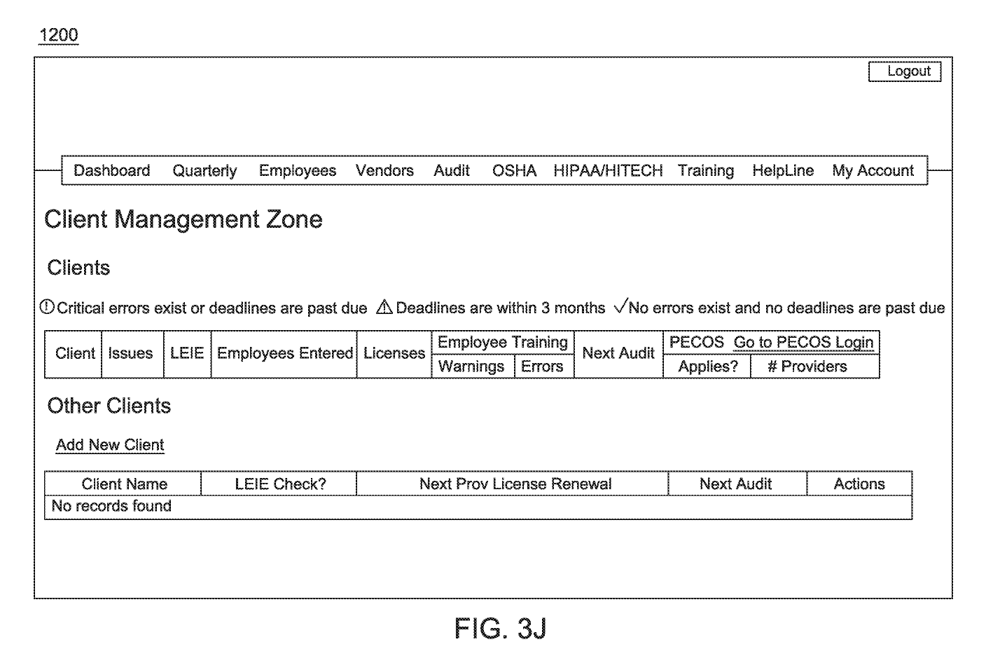

FIGS. 3A-3J are a series of screenshots 300-1200 of a graphical user interface for a system for managing and monitoring a compliance program for a healthcare entity, according to embodiments. The screenshots illustrate aspects of the healthcare compliance portal 102 and the compliance zones in which various regulations are organized. As will be appreciated by those of skill in the art, login and password information may be established and saved, allowing various users access to the portal 102.

Screenshot 300, shown in FIG. 3A, illustrates a “Dashboard” as seen by a supervisor 110 and/or a manager 120 on a respective GUI 112 and/or 122. The “Dashboard” provides an overview of the compliance standards and status for a particular healthcare entity, allowing the supervisor 110 and/or a manager 120 to easily identify those areas in which compliance standards are satisfied and those that need to be satisfied.

Screenshot 400, shown in FIG. 3B, illustrates a “Quarterly Compliance Zone” as seen by a supervisor 110 and/or a manager 120 on a respective GUI 112 and/or 122. The “Quarterly Compliance Zone” presents a series of queries, or questions, to the user, and allows for entry and submission of responses to the queries, which are transmitted to the portal 102 for use in evaluating compliance and preparing compliance profiles and/or reports. The “Quarterly Compliance Zone” also provides a summary of the status, allowing supervisor 110 and/or a manager 120 to obtain an overview of the quarterly compliance efforts.

Screenshot 500, shown in FIG. 3C, illustrates an “Employee Compliance Zone” as seen by a supervisor 110 and/or a manager 120 on a respective GUI 112 and/or 122. The “Employee Compliance Zone” provides a summary and status of compliance issues for all of the employees, as well as a summary and status for each of the employees. Additional details relating to the employees may be provided by selecting (e.g., clicking) on a name of a particular employee of interest, which will result in a screenshot with details relating to the selected employee.

Screenshot 600, shown in FIG. 3D, illustrates a “Vendor Compliance Zone” as seen by a supervisor 110 and/or a manager 120 on a respective GUI 112 and/or 122. The “Vendor Compliance Zone” provides a summary and status of compliance issues for all of the vendors, as well as a summary and status for each of the vendors. Additional details relating to the vendors may be provided by selecting (e.g., clicking) on a name of a particular vendor of interest, which will result in a screenshot with details relating to the selected vendor. An “offshore functions” section provides an alert identifying issues that are outstanding and need to be resolved.

Screenshot 700, shown in FIG. 3E, illustrates an “Audit Compliance Zone” as seen by a supervisor 110 and/or a manager 120 on a respective GUI 112 and/or 122. The “Audit Compliance Zone” allows a user to create an audit entry and view audit history for the healthcare entity.

Screenshot 800, shown in FIG. 3F, illustrates an “OSHA Compliance Zone” as seen by a supervisor 110 and/or a manager 120 on a respective GUI 112 and/or 122. The “OSHA Compliance Zone” includes a summary and status for a given period, indicating compliance with OSHA regulations. The “OSHA Compliance Zone” also includes a series of questions or queries to be entered by a user, for determining compliance with OSHA regulations for the healthcare entity.

Screenshot 900, shown in FIG. 3G, illustrates a “HIPAA/HITECH Compliance Zone” as seen by a supervisor 110 and/or a manager 120 on a respective GUI 112 and/or 122. The “HIPAA/HITECH Compliance Zone” includes a summary and status for a given period, indicating compliance with HIPAA/HITECH regulations. The “HIPAA/HITECH Compliance Zone” also includes a series of questions or queries to be entered by a user, for determining compliance with HIPAA/HITECH regulations for the healthcare entity.

Screenshot 1000, shown in FIG. 3H, illustrates a “Training and Education Compliance Zone” containing training resources, training status, and a summary of training for employees.

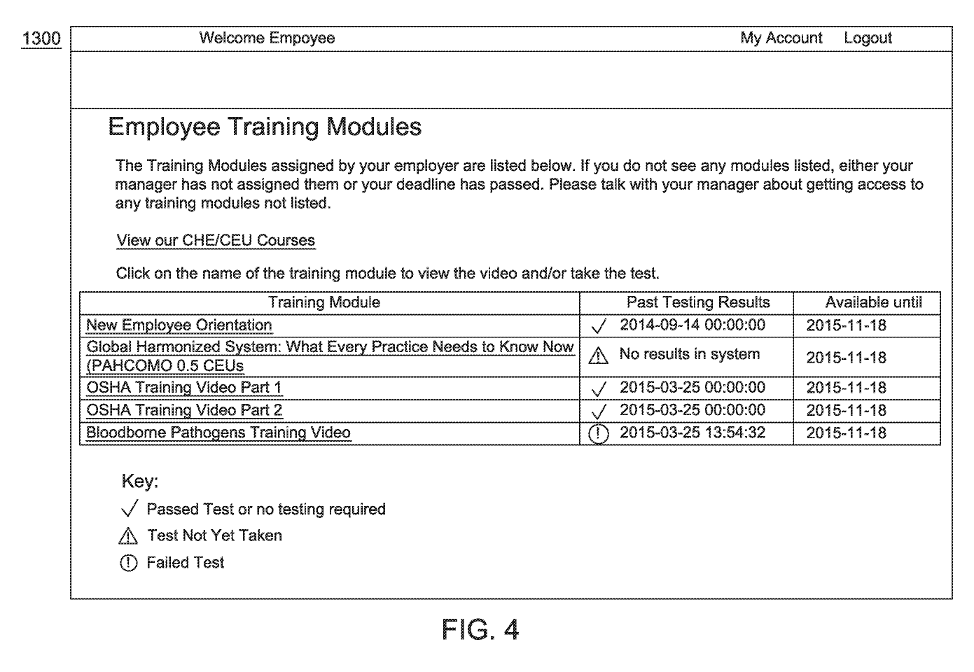

Screenshot 1100, shown in FIG. 3I, illustrates a “Helpline Compliance Zone” for creating and viewing helpline entries. In an embodiment, the “Helpline Compliance Zone” or “Premium Zone” allows a supervisor 110 and/or a manager 120 to create and set a message that appears to all or a subset of employees 130 when logged into their respective employee portal (see FIG. 4 and its accompanying description). In an additional embodiment, the “Helpline Compliance Zone” may also allow for a supervisor 110 and/or a manager 120 to directly email employees 130 (e.g., those employees of a specific location or department).

Screenshot 1200, shown in FIG. 3J, illustrates a “Client Management Zone” as seen by an entity, on a GUI, that manages or oversees one or more clients. The “Client Management Zone” allows the entity to identify compliance issues for the one or more clients. As described above, in an embodiment, a billing entity may utilize the healthcare compliance portal 102 to determine if one or more healthcare entities are in compliance with all or a subset of applicable compliance standards.

FIG. 4 is a screenshot 1300 of an exemplary employee GUI 132, according to an embodiment. The screenshot 1300 includes various training modules for a particular employee 130. Selection of one or more of the training modules by the employee 130 provides for the employee to view relevant training and/or participate in various exercises to establish compliance.

According to an embodiment, also provided in various ones of screenshots 300-1300 are various resources, such as articles and documents, as well as messages, each relevant and tailored to the specific user currently utilizing the portal 102 and/or the compliance zone being viewed.

As will be understood by one of ordinary skill in the art, the screenshots 300-1300 of a graphical user interface are exemplary and may be modified based on the compliance program being implemented and based on details of the particular healthcare entity.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a head disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network (LAN), a wide area network (WAN) and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including LAN or WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatuses (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be appreciated that the above figures and description provide exemplary, non-limiting configurations. Although the present invention has been described with reference to these exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

1. A system for managing and monitoring a compliance program for a healthcare entity, the healthcare entity comprised of (i) one or more supervisors, (ii) one or more employees, and (iii) one or more vendors, the one or more employees and the one or more vendors required to comply with one or more compliance standards, the system comprising:
a portal comprising one or more servers operating over a network configured to interconnect one or more of the one or more supervisors, the one or more employees, and the one or more vendors;
wherein the one or more servers are configured to:
receive input relating to one or more of employee data and vendor data;
receive input relating to the one or more compliance standards;
compile a compliance profile for each of the one or more employees and the one or more vendors for whom input is received; and
convey, through a graphical user interface (GUI), the compliance profile to the one or more supervisors and a corresponding one of the one or more employees and the one or more vendors, the compliance profile indicating compliance measures for the one or more compliance standards applicable to the one of the one or more employees and the one or more vendors.

2. The system of claim 1, wherein the one or more servers are further configured to:
generate one or more queries relating to the one or more compliance standards for the one of the one or more employees and the one or more vendors;
convey, through the GUI, the one or more queries, to the one of the one or more employees and the one or more vendors;
receive inputs in response to the one or more queries;
compile an updated compliance profile for the one of the one or more employees and the one or more vendors; and
convey, through the GUI, the updated compliance profile to the one or more supervisors and the one of the one or more employees and the one or more vendors, the updated compliance profile indicating updated compliance measures for the one or more compliance standards applicable to the one of the one or more employees and the one or more vendors.

3. The system of claim 1, further comprising:
one or more databases for storing data relating to one or more of (i) the employee data, (ii) the vendor data, (iii) the input relating to the one or more compliance standards, and (iv) the compliance profiles;
wherein the data contained on the one or more databases is accessible by the one or more servers.

4. The system of claim 1, wherein the one or more servers are further configured to:
compile a healthcare entity compliance profile for the healthcare entity comprised of the one or more employees and the one or more vendors;
convey, through the GUI, the healthcare entity compliance profile to the one or more supervisors, the healthcare entity compliance profile indicating a summary of compliance measures for the one or more compliance standards applicable to the one or more employees and the one or more vendors.

5. The system of claim 4, wherein the one or more servers are further configured to:
convey the healthcare entity compliance profile to one or more billing entities, wherein the conveyance is through one or more of an electronic message, a dedicated billing GUI, and a secure URL link.

6. The system of claim 4, wherein the conveyance of the healthcare entity compliance profile is performed at a predetermined interval.

7. The system of claim 1, wherein the one or more servers are further configured to at least one of:
convey, through an employee GUI, supplemental employee training materials; and
convey, through a vendor GUI, supplemental vendor training materials.

8. The system of claim 1, wherein the GUI comprises one or more of a supervisor GUI, an employee GUI, and a vendor GUI.

9. The system of claim 1, wherein the one or more servers are further configured to:
access one or more external databases to compare employee data with government data as part of the compliance profile for each of the one or more employees.

10. A system for managing and monitoring a compliance program for a healthcare entity, the healthcare entity comprised of (i) one or more supervisors, (ii) one or more employees, and (iii) one or more vendors, each required to comply with one or more compliance standards, the system comprising:
a portal comprising one or more servers operating over a network configured to interconnect one or more of the one or more supervisors, the one or more employees, and the one or more vendors;
wherein the one or more servers are configured to:
receive input relating to one or more of healthcare entity data, employee data, and vendor data;
receive input relating to the one or more compliance standards from a system administrator;
generate one or more queries for each of the one or more supervisors, the one or more employees, and the one or more vendors, the queries relating to the one or more compliance standards for corresponding ones of the one or more supervisors, the one or more employees, and the one or more vendors;
receive inputs in response to the one or more queries from the one or more supervisors, the one or more employees, and the one or more vendors;
compile a healthcare entity compliance profile comprising a summary of compliance measures for the one or more compliance standards applicable to the one or more supervisors, the one or more employees, and the one or more vendors; and
convey, through a graphical user interface (GUI), the healthcare entity compliance profile to the one or more supervisors.

11. A computer-implemented method for managing and monitoring a compliance program for a healthcare entity, the healthcare entity comprised of (i) one or more supervisors, (ii) one or more employees, and (iii) one or more vendors, the one or more employees and the one or more vendors required to comply with one or more compliance standards, wherein the one or more supervisors, the one or more employees, and the one or more vendors form a network interconnected to one or more servers comprising a portal, the method comprising:
receiving, by the one or more servers, input relating to employee data and vendor data;
receiving, by the one or more servers, input relating to the one or more compliance standards;
compiling, by the one or more servers, a compliance profile for each of the one or more employees and the one or more vendors; and
conveying, by the one or more servers through a graphical user interface (GUI), the compliance profile to the one or more supervisors and a corresponding one of the one or more employees and the one or more vendors, the compliance profile indicating compliance measures for the one or more compliance standards applicable to the one of the one or more employees and the one or more vendors.

12. The method of claim 11, further comprising:
generating, by the one or more servers, one or more queries relating to the one or more compliance standards for the one of the one or more employees and the one or more vendors;
conveying, by the one or more servers through the GUI, the one or more queries, to the one of the one or more employees and the one or more vendors;
receiving, by the one or more servers, inputs in response to the one or more queries;
compiling, by the one or more servers, an updated compliance profile for the one of the one or more employees and the one or more vendors; and
conveying, by the one or more servers through the GUI, the updated compliance profile to the one or more supervisors and the one of the one or more employees and the one or more vendors, the updated compliance profile indicating updated compliance measures for the one or more compliance standards applicable to the one of the one or more employees and the one or more vendors.

13. The method of claim 11, wherein one or more databases for storing data relating to one or more of (i) the employee data, (ii) the vendor data, (iii) the input relating to the one or more compliance standards, and (iv) the compliance profiles, are accessible by the one or more servers.

14. The method of claim 11, further comprising:
compiling, by the one or more servers, a healthcare entity compliance profile for the healthcare entity comprised of the one or more employees and the one or more vendors; and
conveying, by the one or more servers through the GUI, the healthcare entity compliance profile to the one or more supervisors, the healthcare entity compliance profile indicating a summary of compliance measures for the one or more compliance standards applicable to the one or more employees and the one or more vendors.

15. The method of claim 14, further comprising:
conveying, by the one or more servers, the healthcare entity compliance profile to one or more billing entities, wherein the conveyance is through one or more of an electronic message, a dedicated billing GUI, and a secure URL link.

16. The method of claim 14, wherein the conveyance of the healthcare entity compliance profile is performed at a predetermined interval.

17. The method of claim 11, further comprising:
conveying, by the one or more servers through an employee GUI, supplemental employee training materials; and
conveying, by the one or more servers through a vendor GUI, supplemental vendor training materials.

18. The method of claim 11, wherein the GUI comprises a supervisor GUI, an employee GUI, and a vendor GUI.

19. The method of claim 11, further comprising:
accessing, by the one or more servers, one or more external databases to compare employee data with government data as part of the compliance profile for each of the one or more employees.

20. A system for monitoring a compliance program by a billing entity for a healthcare entity comprised of (i) one or more supervisors, (ii) one or more employees, and (iii) one or more vendors, each required to comply with one or more compliance standards, the system comprising:
a portal comprising one or more servers operating over a network configured to interconnect one or more of the one or more supervisors, the one or more employees, and the one or more vendors;
wherein the one or more servers are configured to:
compile a compliance profile for each of the one or more employees and the one or more vendors, the compliance profile indicating compliance measures for the one or more compliance standards applicable to the one of the one or more employees and the one or more vendors;
compile a healthcare entity compliance profile for the healthcare entity, the healthcare entity compliance profile indicating a summary of the compliance measures for the one or more compliance standards applicable to the one or more employees and the one or more vendors and compliance issues for the healthcare entity; and
convey, through a graphical user interface (GUI), to the billing entity one or more of (i) the compliance profile for each of the one or more employees and the one or more vendors and (ii) the healthcare entity compliance profile.
